(19) United States
(12) Patent Application Publication
(10) Pub. No.: US 2023/0270915 A1
(43) Pub. Date: Aug. 31, 2023

(54) CALCIUM-DEFICIENT SILICATE-SUBSTITUTED CALCIUM PHOSPHATE APATITE COMPOSITIONS AND METHODS

(71) Applicant: OSSDSIGN AB, Uppsala (SE)

(72) Inventors: Iain GIBSON, Dundee (GB); Jordan CONWAY, Dundee (GB)

(21) Appl. No.: 18/040,074

(22) PCT Filed: Aug. 6, 2021
(86) PCT No.: PCT/IB2021/057277

§ 371 Date: Jan. 31, 2023

(30) Foreign Application Priority Data
Aug. 7, 2020 (GB) 2012350.1

(51) Int. Cl. A61L 27/12; A61L 27/56; A61L 27/32; C01B 33/36

(52) U.S. Cl. CPC A61L 27/12; A61L 27/56; A61L 27/32; C01B 33/36; A61L 2430/02; C01P 2002/50; C01P 2006/12; C01P 2004/61; C01P 2002/72; C01P 2004/03

(57) ABSTRACT

A calcium-deficient silicate-substituted calcium phosphate apatite composition comprises an apatite phase having a Ca/P molar ratio of from greater than 2.15 to 2.30, and a Ca/(P+Si) molar ratio of from 1.45 to 1.55. A method of producing a calcium-deficient silicate-substituted calcium phosphate apatite composition comprises contacting a silicate-substituted calcium phosphate apatite starting material with an acidic solution to produce the calcium-deficient silicate-substituted calcium phosphate apatite composition. The starting material comprises an apatite phase and up to 15 wt % total of a phase or phases other than the apatite phase, and has a Ca/P molar ratio of from 2.3 to 2.6, and a Ca/(P+Si) molar ratio of from 1.56 to 1.66, and the calcium-deficient silicate-substituted calcium phosphate apatite composition comprises an apatite phase having a Ca/P molar ratio lower than the Ca/P ratio of the starting material apatite phase.

### FIELD OF THE INVENTION

The present invention relates to calcium phosphate materials, particularly materials useful in body tissue repair, principally bone repair and bone replacement, and also to processes for the preparation of such calcium phosphate materials.

### BACKGROUND

Due to disease or trauma, surgeons need to replace bone tissue. They can use bone grafts (autografts or allografts) or synthetic materials to replace bone during surgery. Amongst the types of synthetic materials used to replace bone, surgeons use metals (e.g. stainless steel hip or knee implants), polymers (e.g. polyethylene in acetabular cups), ceramics (e.g. hydroxyapatite as a macroporous bone graft) or inorganic-organic composites (e.g. hydroxyapatite-poly(lactic acid) composites for fixation plates).

Calcium phosphate ceramics, such as hydroxyapatite or tricalcium phosphate, are commonly utilised as bone graft materials and are typically produced by forming a macroporous structure, similar to that of cancellous bone. Such bone grafts typically have large values of total porosity (60-90%) with the porosity existing as a mixture of macropores (0.1 to 1 mm in size) and micropores (0.5 to 10 μm), with the macropores interconnected. These materials usually require high sintering temperatures, typically between 1100-1300° C., as part of the manufacturing process, to densify the calcium phosphates making up the porous ‘cancellous’ structure. Such a macroporous bone graft, typically used in granular form, is classed as osteoconductive, meaning that it acts as a scaffold and allows bone to grow along its surface. Unlike autografts, most synthetic calcium phosphate bone grafts are not osteoinductive. Osteoinductivity is the ability to induce new bone formation by directing undifferentiated mesenchymal stem cells to differentiate and form bone. Recently, some groups have reported developments of synthetic bone grafts based on calcium phosphates that are osteoinductive. The accepted test for osteoinductivity is the implanting of the bone graft material in a non-osseous (non-bone) site, either subcutaneously or intramuscularly in a suitable animal model, and, using histology and histomorphometry, determining if bone is formed in this site. A bone graft material that is only osteoconductive does not form bone in this site, whereas an osteoinductive material does form bone. The advantage of an osteoinductive bone graft is that, when implanted into a bone defect in humans, it will have an accelerated rate of bone repair because bone can form at the interface of the implant and host bone by an osteoconductive response, and also throughout the implant by an osteoinductive response. For new bone to form throughout an osteoconductive bone graft requires a longer time after implantation, as new bone migrates throughout the bone graft from the interface of the implant and host bone.

When calcium phosphates are used to apply coatings on the surface of bioinert implant materials, such as metals or polymers, the calcium phosphate, typically hydroxyapatite, is thermally sprayed onto the metallic or polymeric substrate. This thermal spraying process involves passing the calcium phosphate in powder form into a high temperature flame, typically a plasma flame, and spraying this onto the substrate. This requires the calcium phosphate material to have good thermal stability to avoid complete melting as it passes through the high temperature flame, as otherwise the coating will be composed of large quantities of amorphous phase and/or impurity phases that can affect the ability of the coating to adhere to the substrate and to integrate with surrounding bone on implantation. Some materials are not compatible with this process as they are not thermally stable at the temperatures used in the thermal spraying process. The material may undergo melting or phase decomposition leading to the presence of impurity phases within the coating.

WO 2010/079316 describes osteoinductive calcium phosphate materials with a Ca/P molar ratio in the range of 2.05 to 2.55 and a Ca/(P+Si) molar ratio that is less than 1.66. The materials are unsintered, having only been thermally treated at low temperatures, typically 900° C. However, although these materials offer an improvement over the prior art based on their osteoinductivity and high solubility, the materials undergo phase decomposition at relatively low temperature. At temperatures between 950-1050° C. these compositions start to decompose with the formation of impurity phases, with the amount and number of impurity phases increasing significantly as these temperatures are increased further. This means that these types of compositions are not suitable for use to make macroporous ceramic bone graft substitutes (which requires sintering at temperatures >1100° C.) or for applying thermally sprayed coatings to medical devices.

It would therefore be desirable to prepare alternative materials which would provide the benefits of the materials described in WO 2010/079316 (including, for example, osteoinductivity), while simultaneously being more suitable for use in applications which require the materials to be exposed to relatively high temperatures, including the manufacture of macroporous ceramic bone graft substitutes or the coating of medical devices. The present invention has been developed with a view to addressing this problem.

### SUMMARY OF THE INVENTION

At its most general, the present invention relates to temperature-resistant calcium-deficient silicate-substituted apatite-like compositions and processes for preparation of such compositions. The inventors have found that by contacting certain silicate-substituted apatite-like compositions with acidic solutions, it is possible to prepare derivative materials which exhibit an increased thermal stability. The derivative materials are not only more thermally stable, but they also exhibit osteoinductive properties when implanted in the body, for example as a coating on a medical device or in a granular form.

According to a first aspect of the present invention, there is provided a calcium-deficient silicate-substituted calcium phosphate apatite composition comprising an apatite phase having a Ca/P molar ratio of from greater than 2.15 to 2.30, and a Ca/(P+Si) molar ratio of from 1.45 to 1.55.

In a second aspect of the present invention there is provided a method of producing a calcium-deficient silicate-substituted calcium phosphate apatite composition, comprising contacting a silicate-substituted calcium phosphate apatite starting material with an acidic solution to produce the calcium-deficient silicate-substituted calcium phosphate apatite composition. The starting material comprises an apatite phase and has a Ca/P molar ratio of from 2.3 to 2.6, and a Ca/(P+Si) molar ratio of from 1.56 to 1.66. The calcium-deficient silicate-substituted calcium phosphate apatite composition comprises an apatite phase having a Ca/P molar ratio which is lower than the Ca/P ratio of the starting material apatite phase before contact with the acidic solution.

The compositions of the invention have a chemical composition which differs from that of the starting material. Without wishing to be bound by theory, it is believed that contacting the silicate-substituted calcium phosphate apatite phase starting material with the acidic aqueous solution causes a change in the chemical composition of the silicate-substituted calcium phosphate apatite phase, specifically causing a reduction in the Ca/P molar ratio and the Ca/(P+Si) molar ratio of the silicate-substituted calcium phosphate apatite phase to provide a calcium-deficient silicate-substituted calcium phosphate apatite composition with an apatite phase which has a higher thermal stability. Also without wishing to be bound by theory, it is believed that the silicate content of the apatite phase of the product composition does not change significantly during the process. It has been shown that the compositions which result from this method have osteoinductive properties and furthermore are thermally stable at relatively high temperature, for example temperatures of around 1200° C. In particular it has been found that the method produces compositions which do not undergo phase change when subjected to high temperature, maintaining a hydroxyapatite-like crystal phase and thereby enabling the use of the compositions in applications which require exposure to high temperatures, for example, in the manufacture of macroporous ceramic bone graft substitutes or the coating of medical devices.

The method thereby provides a means to improve the thermal stability of silicate-substituted calcium phosphate apatite phase-containing materials.

A third aspect of the invention is a calcium-deficient silicate-substituted calcium phosphate apatite composition obtained or obtainable by a method according to the second aspect.

The calcium-deficient silicate-substituted calcium phosphate apatite compositions of the first and third aspects have a higher thermal stability than known silicate-substituted calcium phosphate apatite materials, while retaining osteoinductive properties.

A fourth aspect of the invention is a calcium-deficient silicate-substituted calcium phosphate apatite composition according to the first or third aspect, for use in a method of treatment of the human or animal body by surgery or therapy.

A fifth aspect of the invention is a method of treatment of a patient using the calcium-deficient silicate-substituted calcium phosphate apatite composition according to the first or third aspect.

A sixth aspect of the invention is a medical device comprising a coating which includes a calcium-deficient silicate-substituted calcium phosphate apatite composition of the first or third aspect.

A seventh aspect of the invention is a macroporous ceramic bone graft substitute comprising a calcium-deficient silicate-substituted calcium phosphate apatite composition of the first or third aspect.

An eighth aspect of the invention is the use of an acidic solution to improve the thermal stability of a silicate-substituted calcium phosphate apatite composition.

A ninth aspect of the invention is a method of improving the thermal stability of a silicate-substituted calcium phosphate apatite phase starting material, comprising contacting the silicate-substituted calcium phosphate apatite phase starting material with an acidic solution. The starting material has a Ca/P molar ratio of from 2.3 to 2.6, and a Ca/(P+Si) molar ratio of from 1.56 to 1.66.

A tenth aspect of the invention is a method of manufacturing a medical device for use as an implant, comprising applying a calcium-deficient silicate-substituted calcium phosphate apatite composition of the first or third aspect to a bioinert substrate.

### DETAILED DESCRIPTION

A first aspect of the invention is a calcium-deficient silicate-substituted calcium phosphate composition comprising an apatite phase having a Ca/P molar ratio of from greater than 2.15 to 2.30, and a Ca/(P+Si) molar ratio of from 1.45 to 1.55. In a second aspect of the present invention there is provided a method of producing a calcium-deficient silicate-substituted calcium phosphate apatite composition. The method comprises contacting a silicate-substituted calcium phosphate apatite phase starting material with an acidic solution, wherein the starting material comprises an apatite phase and has a Ca/P molar ratio of from 2.3 to 2.6, and a Ca/(P+Si) molar ratio of from 1.56 to 1.66.

The silicate-substituted calcium phosphate apatite starting material (referred to as “starting material” herein) may be any suitable silicate-substituted calcium phosphate apatite comprising an apatite phase and having a Ca/P molar ratio of from 2.3 to 2.6, and a Ca/(P+Si) molar ratio of from 1.56 to 1.66.

The starting material may comprise an inorganic compound comprising calcium ions, phosphate ions (PO₄³⁻) and hydroxyl ions (OH⁻), along with silicate ions (SiO₄⁴⁻). The starting material may be a synthetic inorganic compound. The starting material may be an inorganic crystalline or semi-crystalline material comprising calcium ions, phosphate ions (PO₄³⁻) and hydroxyl ions (OH⁻), along with silicate ions (SiO₄⁴⁻).

In some embodiments the starting material is in granular or particulate form. The starting material may be a powder. In some embodiments the starting material comprises granules.

In some embodiments the starting material comprises a silicate-substituted hydroxyapatite. For clarification, the incorporation of silicon into an apatite or hydroxyapatite material can be referred to as silicon or silicate substitution. These terms can be used interchangeably. This is because the substitution is actually of a silicon atom substituting for a phosphorus atom in the apatite or hydroxyapatite lattice (strictly, the Si or P exists in the structure as an ion and not as a neutral atom). The phosphorus or silicon atom is, however, always associated with oxygen to form phosphates or silicates which in the invention may be, for example, but are not limited to, SiO₄⁴⁻ or PO₄³⁻ ions. The starting material may be an inorganic silicate-substituted calcium phosphate hydroxyapatite.

In some embodiments, the starting material may be represented by formula (I):

Ca_{10-δ}(PO₄)₆₋ₓ(SiO₄)ₓ(OH)_{2-y} (I)

wherein 1.1≤x≤2.0, 1.0≤y≤2.0, and 6 represents a Ca deficiency such that the Ca/(P+Si) molar ratio has a value of from 1.56 to 1.66. Preferably 1.2≤x≤2.0, more preferably 1.4≤x≤2.0 and most preferably 1.6≤x≤2.0. Generally, it is desirable that the compound contains hydroxyl ions. The amount of hydroxyl ions, represented by y in formula (I), can be controlled by the levels of x and 6, but also independently by thermal heat treatment, so it is considered a somewhat independent variable.

Most preferably, 1.5≤x≤2.0, for example 1.6≤x≤2.0. Most preferably, 1.5≤x≤2.0 and 6=0, for example 1.6≤x≤2.0 and δ=0.

In some embodiments, the starting material has a silicon atom content of 4 to 6 wt %.

The starting material may have a high level of silicon incorporated into a hydroxyapatite phase that also contains calcium, phosphorus, oxygen and hydrogen ions, more specifically calcium, phosphate and hydroxyl ions. They may have a hydroxyapatite structure, and preferably have sub-micron crystal morphology, in which case they are not classed as a ceramic or bioceramic which have monolithic structures and consist of fused grain structures that are separated by grain boundaries. The starting material is preferably in the form of an unsintered material. This is achieved by heating the material at temperatures below the typical sintering temperature of hydroxyapatites during synthesis, so that sintering does not occur. Furthermore, the starting material has a tendency to be thermally unstable at high temperatures; therefore it is preferably used as a powder or compacted powder, and preferably not fused in the manner of sintered ceramic hydroxyapatites.

The silicon atom content of the starting material is preferably at least 2.9 wt %, more preferably at least 3.5 wt %, and most preferably at least 4 wt %. These values are equivalent to a silicate (SiO₄) content of at least 9.5 wt %, at least 11.5 wt %, and at least 13 wt % respectively. A higher silicon content carries through to the calcium-deficient products of the invention and, in use, is desirable to release a larger amount of silicon when the compositions are immersed in solution, in particular for biomedical applications used in bone formation and bone metabolism. Also, the properties of the hydroxyapatite are believed to change at a silicon atom content in the region of 2.9 wt % (9.5 wt % silicate) or above. The maximum silicon atom content of the starting material is preferably 6 wt % (20 wt % silicate). The silicon atom content is preferably in the range 3.5 to 6 wt % (11.5 to 20 wt % silicate), and more preferably in the range 4 to 6 wt % (13 to 20 wt % silicate).

The molar ratio of calcium to phosphorus-containing ions (Ca/P molar ratio) in the starting material is higher than that observed in stoichiometric hydroxyapatite (which is 10:6, or 1:0.6, or a Ca/P molar ratio of 1.67). Accordingly, in one embodiment, the Ca/P molar ratio of the starting material is at least 2.3, and more specifically from 2.3 to 2.6.

The starting material has a Ca/(P+Si) molar ratio of from 1.56 to 1.66. The molar ratio of Ca/(P+Si) may be less than 1.66, or not more than 1.65. Preferably the Ca/(P+Si) molar ratio is in the range 1.56 to 1.65, more preferably in the range 1.60 to 1.65, yet more preferably in the range 1.60 to 1.64.

Higher Ca/P molar ratios than 2.55 will result in additional phases being present. Accordingly, in specific embodiments, the Ca/P molar ratio may be in the range 2.3 to 2.55, preferably 2.3 to 2.5. Preferably the starting material is free of carbonate ions (CO₃) in the apatite structure. The maximum impurity level of carbonate ions is preferably 1.0%, more preferably 0.5%, more preferably 0.1%, more preferably 0.01%, as a molar ratio based on the total of silicate and phosphoric ions. Thus carbonate substitution for phosphate (or silicate) in the starting material may be substantially absent.

Preferably, the starting material is in crystalline form, particularly polycrystalline, e.g. polycrystalline particles. In some specific embodiments, the crystallite average long-axis length may be 5 μm or less for improved solubility, and is preferably at least 0.05 μm. Preferably, the crystallite long-axis length is in the range 0.05 to 5 μm.

The starting material comprises a silicate-substituted calcium phosphate apatite phase, i.e. a single phase of apatite material. In some embodiments, the starting material comprising the silicate-substituted calcium phosphate apatite phase contains other phases, for example impurity phases. In specific embodiments, the starting material contains not more than 15 wt % total of one or more impurity phases. Within the present disclosure, phase impurity is determined by X-ray diffraction according to ASTM F2024—Standard Practice for X-ray Diffraction Determination of Phase Content of Plasma-Sprayed Hydroxyapatite Coatings. A composition of a hydroxyapatite composition is generally defined as a phase composition of at least 95 wt % hydroxyapatite phase, with up to a maximum of 5 wt % phase impurity (ASTM F1185—Standard Specification for Composition of Hydroxylapatite for Surgical Implants). The present invention can use a starting material that would be within this specification, having not more than 5 wt % phase impurity, but may also employ a starting material that falls outside this specification, with greater than 5 wt %, but not more than 15 wt % impurity phase(s). Therefore, in some embodiments, the material may have one impurity phase, i.e., being biphasic, or may have more than one impurity phase, i.e., being multiphasic. In other embodiments, the material comprising the silicate-substituted calcium phosphate apatite phase does not contain other phases, i.e. the material is phase pure. It is expected that the method of the invention will result in a change in chemical composition which is not strongly dependent on the initial phase composition. For example, a silicate-substituted hydroxyapatite starting material with a small (not more than 15 wt %) phase impurity of tricalcium phosphate and/or of calcium oxide can be subjected to the same controlled immersion process as a single phase silicate-substituted hydroxyapatite starting material with a comparable new composition obtained. In specific embodiments, the inventive calcium-deficient silicate-substituted calcium phosphate apatite composition contains not more than 5 wt % total of one or more impurity phases. In more specific embodiments, the inventive calcium-deficient silicate-substituted calcium phosphate apatite composition consists or consists essentially of the apatite phase, i.e., no detectable amount or no materially effecting amount, respectively, of phases other than the apatite phase are present.

In specific embodiments, the starting material is substantially phase pure. This means that there are substantially no impurity phases. So, for example, only one polycrystalline phase may be seen by X-ray diffraction, with no secondary phases visible in the diffraction pattern. The presence of a single silicate-substituted hydroxyapatite phase can be determined using conventional X-ray diffraction analysis and comparing the obtained diffraction pattern with standard patterns for hydroxyapatite. The exact diffraction peak positions of the silicate-substituted hydroxyapatite phase show a small shift compared to the diffraction peak positions of a hydroxyapatite standard, as the substitution of silicate for phosphate results in a change in the unit cell parameter. This has previously been reported for small amounts of silicate substitution (e.g. I. R. Gibson et al, J. Biomed. Mater. Res. 44 (1999) 422-428). The amount of silicon or silicate incorporated into a silicate-substituted hydroxyapatite, and the Ca/P molar ratio of a silicate-substituted hydroxyapatite may also be evaluated using chemical analysis techniques, for example, X-Ray Fluorescence (XRF). The starting material may be characterised by having a molar ratio as determined by XRF of Ca/(P+Si) of from 1.56 to 1.66, from 1.56 to less than 1.66, preferably not more than 1.65, e.g. not more than 1.64, and a Ca/P molar ratio of from 2.3 to 2.6. Within the present disclosure, XRF is suitably performed with the formation of lithium borate glass fluxes with the material to be tested according to ISO 12677:2011—Chemical analysis of refractory products by X-ray fluorescence (XRF), Fused cast-bead method.

Suitable silicate-substituted calcium phosphate apatite phase-containing materials are described in U.S. Pat. Nos. 8,545,895 and 9,492,585 (the contents of which are incorporated herein by reference in their entirety), however the starting material is not limited to the compositions described therein.

The starting material may have been subjected to one or more thermal processing treatments after its synthesis and before the method of the second aspect of the invention, although this is not necessary and the starting material may simply have been synthesised (e.g. by a precipitation reaction as set out in one of the patents mentioned above) and isolated from the reaction mixture by e.g. filtering and drying, without any high temperature treatment steps. Possible heat treatment steps prior to the method of the invention include calcining the material at a temperature of at least 700° C., for example at least 750° C., at least 800° C. or at least 850° C., for example from 700 to 1000° C., from 750 to 950° C., or from 800 to 950° C.

Where such a heat treatment step is carried out and the starting material is in powder form, preferably the specific surface area of the starting material powder after heating is in the range 10 to 90 m²/g, more preferably between 20 and 50 m²/g. The specific surface area may be measured by gas adsorption applying the BET theory using the method according to Ph. Eu.2.9.26 Method II.

The physical form of the starting material is not limited, although in some embodiments it may be selected from particle suspension, powder, filter cake, porous granule, porous block, coating or monolithic block.

In some embodiments the starting material is a powder comprising particles having an average particle diameter Dᵥ50 less than 100 μm. Dᵥ50 is the volume median particle diameter and may be determined by laser light scattering according to ASTM B822-17 under the Mie scattering theory, for example using a Malvern Mastersizer 3000.

In some embodiments, the starting material is present within a precipitated suspension comprising the silicate-substituted calcium phosphate apatite phase suspended in a liquid carrier.

In some embodiments the starting material is a granular composition comprising granules having an average particle diameter Dᵥ50 greater than 100 μm. In some embodiments the starting material is a granular composition comprising granules having an average particle diameter Dᵥ50 of from 0.001 to 10 mm, for example from 0.01 to 10 mm, for example from 0.1 to 10 mm, for example from 0.5 to 5 mm, for example from 0.5 to 1.0 mm or from 1.0 to 2.0 mm.

As noted above, the phase composition of the starting material is not limited, and may be selected from a single phase composition to a biphasic or multiphasic composition, with the method of contacting with the acidic solution resulting in a change in the chemical composition that is not strongly dependent on the initial phase composition. For example a silicate-substituted hydroxyapatite starting material with a small phase impurity of tricalcium phosphate or of calcium oxide may be subjected to the same method with a comparable new composition obtained.

The starting material may have been subjected to additional treatment steps prior to contacting with the acidic solution. Such steps include but are not limited to one or more of oven drying, spray drying, calcining or sintering, which may be applied to any of the physical forms of the starting material mentioned above. Thus in some embodiments the method of the first aspect may comprise, prior to contacting the starting material with the acidic solution, subjecting a precursor of the starting material to one or more treatment steps selected from oven drying, spray drying, calcining and sintering. In specific embodiments however, the starting material has not been subjected to sintering in view of the noted phase decomposition which results from high temperature processing of at least some of the starting materials described herein.

In some embodiments, the starting material may have been dried prior to the step of contacting with the acidic solution by exposure to a temperature within the range from room temperature to 200° C. In some embodiments, the starting material has not been exposed to a temperature greater than 200° C. before contacting with the acidic aqueous solution. This may ensure a higher phase purity of the starting material and therefore a higher phase purity of the product. Thus in some embodiments, after synthesis of the starting material (for example, by precipitation as described above), the step of contacting the starting material with the acidic solution is performed without any intervening heat treatment step in which the starting material is exposed to a temperature greater than 200° C.

Nevertheless, in some embodiments the starting material may have been heat-treated at a temperature of greater than 200° C., for example from 200 to 1200° C., prior to the step of contacting the starting material with the acidic aqueous solution.

In some embodiments, the starting material comprises one or more of micropores (0.5 to 10 μm pore diameter) and macropores (0.1 to 1 mm pore diameter), as determined by mercury intrusion porosimetry. In some embodiments, the starting material may have been subjected to a processing step prior to contacting with the acidic aqueous solution to introduce one or more of microporosity and macroporosity.

The method of the invention comprises contacting the silicate-substituted calcium phosphate apatite starting material with an acidic solution.

Preferably, the acidic solution is an aqueous acidic solution.

In some embodiments, before contacting with the starting material, the acidic solution has a pH of from 3 to less than 7, for example from 3 to 6.9, from 3 to 6.8, from 3 to 6.7, from 3 to 6.6 or from 3 to 6.5. More preferably, for increased efficiency of the process, before contacting with the starting material, the acidic solution has a pH of from 3 to 6, for example from 3.2 to 5.8, from 3.3 to 5.7, from 3.4 to 5.6, from 3.5 to 5.5, from 3.6 to 5.4, from 3.7 to 5.3 or from 3.8 to 5.2. If the starting pH of the acidic solution is less than 3, the acidic solution may dissolve all or an excessive portion of the starting material and/or cause formation of acidic calcium phosphates such as brushite. On the other hand, if the starting pH of the acidic solution is 7 or greater, the desired change in chemical composition of the starting material cannot be achieved. An acidic solution pH in the range of from 3 to less than 7 allows a controlled change in the chemical composition of the starting material.

Preferably, before contact with the starting material, the acidic solution has a pH of from 4 to 5, more preferably from 4.2 to 5, from 4.4 to 5, or most preferably from 4.6 to 4.9.

The acidic solution may comprise an acid component and a liquid vehicle, for example a solvent. When the acidic solution is aqueous, the solvent is water. The acidic aqueous solution may comprise an acid component and water. The acidic aqueous solution may consist of an acid component and water.

The acid component of the acidic solution may be any suitable weak or strong acid. Strong acids include hydrochloric acid, nitric acid, sulphuric acid, hydrobromic acid or perchloric acid. Weak acids include ammonium chloride, citric acid, acetic acid, formic acid, benzoic acid, oxalic acid, sulphurous acid, nitrous acid, boric acid or phosphoric acid.

In some embodiments, the acid component is an acid having a pKa of greater than −1.73, for example greater than −1.5, greater than −1.0 or greater than 0. Preferably, the acid component is an acid having a pKa of from 1.0 to 10.0, for example from 1.2 to 10.0, for example from 1.5 to 10.0, for example from 2.0 to 10.0, for example from 3.0 to 10.0, from 4.0 to 10.0 from 5.0 to 10.0, from 6.0 to 10.0 or from 7.0 to 10.0.

The skilled person will understand that the chosen concentration of the acidic solution will depend on the desired pH of the solution and the pKa of the acid component. A stronger acid may require only a relatively low concentration, whereas a weaker acid may require a higher concentration. Generally, the concentration of the acid component in the solution may range from trace levels up to 5 M. In some embodiments, wherein a strong acid is employed, the acid concentration may be in a range of 1 μM to 1 mM, or in a range of 10 μM to 1 mM. For example, when using a strong acid such as hydrochloric acid, lower concentrations, e.g. 10 μM to 1 mM are suitable to achieve a desired rate of composition change and avoiding very low (acidic) final pH values and excessive dissolution of or mass loss from the starting material. In other embodiments, the concentration of the acid component in the solution is from 0.001 M to 5 M, for example from 0.1 M to 2 M.

In some embodiments, the acid component comprises or consists of ammonium chloride, NH₄Cl. In some embodiments, the acidic solution comprises or consists of an ammonium chloride solution, for example an aqueous ammonium chloride solution. The aqueous ammonium chloride solution may have an ammonium chloride concentration of from 0.01% w/v to 15% w/v, for example from 0.5% w/v to 15 % w/v, from 1% w/v to 15% w/v, from 2% w/v to 15% w/v, from 2% w/v to 12% w/v, from 2% w/v to 10 % w/v, from 3% w/v to 10% w/v, from 4% w/v to 10% w/v, from 4% w/v to 8% w/v or from 4% w/v to 6% w/v. Here, “% w/v” indicates the amount of NH₄Cl in grams per 100 mL of solvent, before mixing to form a solution. So, 50 g NH₄Cl added to 1000 mL of distilled water would provide a 5% w/v (0.93 M) solution according to this definition.

In some embodiments the method comprises preparing the acidic solution by mixing the acid component and the solvent. In some embodiments this comprises adding the acid component to the solvent and mixing until fully dissolved. In some embodiments, this comprises mixing the acid component with the solvent in a sufficient quantity to provide a pH within the range specified above.

The method of the first aspect comprises contacting the starting material with the acidic aqueous solution. In some embodiments, the solution is mixed with the starting material in a weight ratio of at least 5:1, for example at least 10:1, for example at least 15:1, at least 20:1, at least 25:1, at least 30:1, at least 35:1, at least 40:1, at least 45:1 or at least 50:1. Such ratios ensure that the contact time required to effect the formation of the calcium-deficient silicate-substituted calcium phosphate apatite phase is not excessive.

In some embodiments, the solution is mixed with the starting material in a weight ratio of from 10:1 to 100:1, for example from 20:1 to 100:1, from 30:1 to 80:1 or from 40:1 to 80:1.

In some embodiments, contacting the starting material with the acidic aqueous solution comprises combining the starting material with the acidic aqueous solution. The starting material may be added to the solution, or vice versa.

In some embodiments, contacting the starting material with the acidic aqueous solution comprises fully immersing the starting material in the acidic aqueous solution, i.e. combining them in such a way that all of the starting material is fully immersed within the acidic aqueous solution.

In some embodiments, the method of the first aspect comprises forming a mixture of the starting material and the acidic aqueous solution and allowing the starting material and the acidic aqueous solution to remain in admixture for a predetermined period of time. In some embodiments, the method of the first aspect comprises forming a mixture of the starting material and the acidic aqueous solution and allowing the starting material and the acidic aqueous solution to remain in admixture for at least 10 mins, for example at least 30 mins, for example at least 40 mins, for example at least 50 mins, for example at least 1 hour, for example at least 1 hour, for example from 10 mins to 500 hrs, for example from 10 mins to 300 hrs, from 10 mins to 250 hrs, from 10 mins to 200 hrs, from 10 mins to 180 hrs, from 10 mins to 150 hrs, from 30 mins to 150 hrs, from 50 mins to 150 hrs, from 1 hr to 150 hrs or from 24 to 120 hrs. In some embodiments the temperature of the mixture during this period of time is at least 20° C., for example at least 25° C.

In some embodiments, the method of the invention comprises incubating the mixture of the starting material and the acidic aqueous solution (hereafter “incubation mixture”). In some embodiments, the incubation comprises heating the incubation mixture and allowing the incubation mixture to remain at an elevated temperature for a predetermined period of time. In some embodiments, the incubation comprises heating the incubation mixture to a temperature T₁ and allowing the incubation mixture to remain at temperature T₁ for a time t₁, wherein T₁ is at least 30° C. and t₁ is at least 10 mins, for example at least 30 mins, for example at least 40 mins, for example at least 50 mins, for example at least 1 hour, for example at least 1 hour. In some embodiments T₁ is from 30° C. to 100° C., for example from 30° C. to 90° C., from 30° C. to 80° C., from 30° C. to 70° C., from 30° C. to 60° C., from 30° C. to 50° C., from 30° C. to 40° C. or from 35° C. to 40° C. In some embodiments t₁ is from 10 mins to 500 hrs, for example from 10 mins to 300 hrs, from 10 mins to 250 hrs, from 10 mins to 200 hrs, from 10 mins to 180 hrs, from 10 mins to 150 hrs, from 30 mins to 150 hrs, from 50 mins to 150 hrs, from 1 hr to 150 hrs or from 24 to 120 hrs. The exact time chosen for t₁ may depend on a number of factors, including the physical form of the starting material (a less porous form or a form with a lower surface area may require additional incubation time), the type of acid (a stronger acid may reduce the incubation time required), the concentration of acid (a higher concentration of acid may reduce the incubation time required) and the temperature T₁.

Most preferably, the incubation comprises heating the incubation mixture to a temperature T₁ and allowing the incubation mixture to remain at temperature T₁ for a time t₁, wherein T₁ is at least 30° C. and t₁ is at least 25 hrs, for example at least 30 hrs, at least 40 hrs, at least 50 hrs, at least 60 hrs or at least 70 hrs. It has surprisingly been found that when such longer incubation periods are employed the product material has a particularly high thermal phase stability. In particular, at such incubation periods it has been found that the single phase nature of the starting material is preserved in the product and in a sintered material when the product is sintered at high temperature (e.g. 1250° C.), with no observable formation of secondary impurity phases when the product and sintered product are examined by XRD. For example, when the starting material comprises a single phase hydroxyapatite-like phase and such longer incubation periods are applied, the calcium-deficient product when sintered at high temperature does not form any observable impurity phases but retains a single phase hydroxyapatite-like phase. The products of the method of the invention are therefore particularly suited to applications which require exposure to high temperatures.

In some embodiments the incubation time t₁ is selected such that the mass loss of the starting material is at least 4%, for example at least 4.5%, for example at least 5%, for example from 4% to 10%, for example from 5% to 8%, wherein mass loss is calculated according to the following equation:

Relative mass loss=(M₀−M_{f})/M₀×100%

wherein M₀ is the mass of starting material, and M_{f} is the mass of product after separation from the incubation mixture and drying, wherein the drying is performed in a drying oven until constant mass is achieved. It has been observed that the mass loss increases with increasing contact time between the starting material and the acidic solution, and that the desired calcium-deficient silicate-substituted calcium phosphate apatite phase product is obtained when the mass loss is in the ranges specified above.

In some embodiments, for example, when the initial pH of the acidic solution is in the range 4 to 5, the incubation time t₁ is selected such that the pH increase of the incubation mixture is at least 40%, for example at least 42%, for example at least 45%, for example from 40% to 80%, for example from 55% to 75%, wherein pH increase is calculated according to the following equation:

Relative pH change=[([pH_{f}]−[pH₀])/pH₀]×100%

wherein pH₀ is the pH of the acidic solution before contacting with the starting material and pH_{f} is the pH of the incubation mixture at the end of the incubation time t₁. It has been observed that the pH change tends to increase with increasing contact time between the starting material and the acidic solution, and that the desired calcium-deficient silicate-substituted calcium phosphate apatite phase product is obtained when the pH change is in the ranges specified above. The final pH of the incubation mixture (as determined by testing the pH of the filtrate after separating the product from the mixture) may be from 7 to 8, for example from 7.2 to 8.0, from 7.3 to 8.0 or from 7.4 to 8.0.

The exact time required to alter the chemical composition of the starting material will be affected by the form that the material is in, and also the type and concentration of acid used.

The incubation mixture may simply be left at temperature T₁ for time t₁ without any stirring or agitation, but in some embodiments agitation of the mixture may be performed for some or all of the incubation period t₁.

Without wishing to be bound by theory, it is believed that the step of contacting the starting material with the acidic solution changes the chemical composition of the starting material, primarily by reducing the Ca/P molar ratio and reducing the Ca/(P+Si) molar ratio, thereby producing the calcium-deficient silicate-substituted calcium phosphate apatite phase product (hereafter “product”).

The magnitude of the change in the chemical composition of the starting material, namely the Ca/P molar ratio and the Ca/(P+Si) molar ratio, may be controlled by a number of factors. These include: the chemical composition of the starting material; the concentration of acid in the acidic solution (and correspondingly the pH of the acidic solution); the quantity of starting material (g) and the volume of the acidic solution (mL) used; the duration of contact of the starting material with the acidic solution; the temperature of the acidic solution; the physical nature of the starting material (slurry, powder, granules, monoliths, coatings), which influences the material properties (surface area, porosity, crystallinity); the thermal history of the starting material (air dried, calcined, sintered), which influences the material properties (surface area, porosity, crystallinity).

After the step of contacting the starting material with the acidic solution, and after any incubation period, the method may comprise separating the product from the acidic solution. This may be achieved by any know solid-liquid separation technique. In some embodiments, centrifugation is used to separate the product from the solution. In some embodiments, the solution is filtered to separate the product from the liquid. The filtration may be performed under vacuum, for example using a Buchner funnel and filter paper. The filtrate may be discarded.

After separation of the product from the solution, the separated product may be subjected to additional processing steps. In some embodiments, after separation of the product the method further comprises one or more rinsing steps, for example comprising rinsing with distilled water. In some embodiments, rinsing is performed to remove all traces of the acidic solution with which the starting material was contacted. In some embodiments, after separation of the product, and after any rinsing steps, the method further comprises one or more drying steps, for example oven drying or spray drying. Drying may be performed at a temperature of at least 50° C., for example at least 60° C., for example from 60 to 100° C. or from 60 to 80° C.

The choice of the drying apparatus (e.g. oven or spray dryer) may depend on inter alia the physical form of the product (powder, granule or monolith) and the required for of the final dried material.

In some embodiments, after separation of the product, and after any rinsing and drying steps, the method further comprises one or more steps of sintering the product. As explained above, due to the acid treatment step of the method, the calcium-deficient product is more thermally stable than known compositions and may therefore be subjected to one or more sintering steps with little or no change in the product chemical composition and little or no change in phase composition. Although some phase decomposition may occur during sintering depending on the nature of the incubation step of the process, the extent of the phase decomposition is significantly reduced relative to the starting material. The calcium-deficient silicate-substituted phosphate materials can advantageously be densified by such sintering, without changing the chemical composition and without changing the phase composition.

In some embodiments the sintering comprises heating the product in a furnace to a temperature of at least 100° C., for example at least 200° C., at least 300° C., at least 400° C., at least 500° C., at least 600° C., at least 800° C., at least 850° C. or at least 900° C. In some embodiments the sintering comprises heating the product in a furnace to a temperature of from 100° C. to 1500° C., for example from 200° C. to 1500° C., from 300° C. to 1500° C., from 500° C. to 1300° C., from 800° C. to 1250° C., from 850° C. to 1250° C., from 900° C. to 1300° C., from 1000° C. to 1300° C., from 1100° C. to 1300° C. or from 1200° C. to 1300° C.

Sintering may be performed (i.e. the material may be held at the above-specified temperature) for a time of at least 30 minutes, for example at least 1 hour, for example 1-2 hours.

The method of the invention converts the silicate-substituted calcium phosphate apatite phase starting material into the calcium-deficient silicate-substituted calcium phosphate apatite phase product through contact with the acidic solution.

In some embodiments, the calcium-deficient silicate-substituted calcium phosphate apatite composition comprises an apatite phase having a Ca/P molar ratio which is lower than the Ca/P ratio of the apatite phase of the starting material before contact with the acidic solution. In some embodiments, the calcium-deficient silicate-substituted calcium phosphate apatite composition comprises an apatite phase having a Ca/(P+Si) molar ratio which is lower than the Ca/(P+Si) ratio of the apatite phase of the starting material before contact with the acidic solution.

The calcium-deficient silicate-substituted calcium phosphate apatite composition comprises an apatite phase having a Ca/P molar ratio of from greater than 2.15 to 2.30, or, more specifically, from greater than 2.15 to less than 2.30, from 2.00 to 2.30, from 2.20 to less 2.30, or from 2.20 to 2.28. Additionally, the apatite phase of the calcium-deficient silicate-substituted calcium phosphate apatite composition comprises a Ca/(P+Si) molar ratio of from 1.45 to 1.55, or, more specifically, from 1.45 to 1.54, or from 1.45 to 1.52. To the extent that the composition contains one or more impurity phases, for example including calcium such as phases of tricalcium phosphate and/or calcium oxide, the molar ratios of the overall composition may be higher than those noted for the apatite phase of the composition. Therefore, in some embodiments, the calcium-deficient silicate-substituted calcium phosphate apatite composition comprises a Ca/P molar ratio of from greater than 2.15 up to 2.35. In some embodiments, the calcium-deficient silicate-substituted calcium phosphate apatite composition comprises a Ca/(P+Si) molar ratio of from 1.45 to 1.60. For calcium-deficient compositions containing little or no impurity phases, the molar ratios for the composition will be the same or substantially the same as those of the apatite phase.

In some embodiments, the calcium-deficient silicate-substituted calcium phosphate apatite composition has a silicon atom content of from 4 to 6 wt %.

The properties of the composition may be controlled by a number of factors, including (a) the chemical composition of the starting material; (b) the physical form that the starting material is in; (c) the composition of the acidic solution, typically the pH; (d) the duration that the starting material is immersed in the acidic solution; and (e) the post-treatment of the product that results from the method, including temperature treatment and the time at temperature.

Some other factors that may have some effect on the change in chemical composition as a result of the method but are not considered to be as significant are: the physical properties of the starting material (namely surface area and porosity), the temperature of the acidic solution, and whether stirring or agitation of the acidic solution is performed during contact with the starting material.

The method of the invention may further comprise applying the calcium-deficient silicate-substituted calcium phosphate apatite composition (hereafter “product”, whether made by the method described herein or otherwise) to a device. In some embodiments this comprises applying a coating comprising the calcium-deficient silicate-substituted calcium phosphate apatite product to the surface of a device, for example a medical device or, more specifically, an implant.

Alternatively the method may further comprise using the calcium-deficient silicate-substituted calcium phosphate apatite product directly to form a device, for example a medical device, for example, a bone graft material or an implant.

The product may be utilised as a medical device or as part of a medical device and may have application in the treatment of bone defects, trauma or other skeletal indications.

The product may be used as a bone graft material for use in bone repair, where it is both osteoconductive and osteoinductive.

The product may be used as a bone graft material by pre-forming the starting material into the desired final form of the graft, such as a granule, subjecting the granules to the method of the invention, then washing and drying the granules. Thus the method may comprise, prior to contacting the starting material with the acidic solution, forming the starting material into a predetermined physical form.

The product may be used as a bone graft material by pre-forming the starting material into the desired final form of the graft, such as a granule, subjecting the granules to the method as described herein, then washing and drying the granules, and then subjecting the granules to a high temperature treatment to modify one or more properties of the granules, for example the phase composition, the surface area or the porosity. For example, the product may be heated to a temperature which provides an apatite phase with a high surface area (>10 m²/g). Another example is heating the product to a higher temperature which provides an apatite phase with a lower surface area (<10 m²/g).

In some embodiments, the starting material is subjected to the method of the invention, either as a particle suspension, powder, filter-cake, porous granule, porous block or monolithic block, to form the product. This may then be processed to form a bone graft material by typical processing methods to produce porous granules or blocks, including subjecting the processed bone graft of the product to a high temperature treatment to modify the properties of the granules or blocks, for example the phase composition, the surface area or the porosity. An example of this is to heat the product in the form of granules to a temperature which provides an apatite phase with a high surface area (>10 m²/g). Another example is to heat the product in the form of granules to a higher temperature which provides an apatite phase with a lower surface area (<10 m²/g). For clarity the porous granules or blocks of the product may contain nano-porosity (typically 0.1 to 100 nm size), micro-porosity (typically 100 nm to 10 μm), macro-porosity (10 μm to 1000 μm) or a mixture of these porosities, as determined by mercury intrusion porosimetry.

The calcium deficient composition of the invention may be used to form a bone graft material in isolation, i.e., without other materials. To alter the properties of the bone graft, however, the product may be combined with other calcium phosphates. For example one or more of octacalcium phosphate, amorphous calcium phosphate, brushite, monetite or tetracalcium phosphate may be combined with the new composition.

In some embodiments the product is a powder comprising particles having an average particle diameter Dᵥ50 less than 100 μm.

In some embodiments the product is a granular composition comprising granules having an average particle diameter Dᵥ50 greater than 100 μm. In some embodiments the starting material is a granular composition comprising granules having an average particle diameter Dᵥ50 of from 0.001 to 10 mm, for example from 0.01 to 10 mm, for example from 0.1 to 10 mm.

Other phases may be added to the product described above when in granule or powder form. Such other phases include but are not limited to calcium carbonate, calcium sulphate, calcium silicate, calcium silicate glass, calcium silicate-based glass, calcium phosphate glass, calcium phosphate-based glass, calcium silicate-based glass-ceramic, calcium phosphate-based glass-ceramic, bioactive glasses, bioactive glass ceramics, biocompatible glasses, biocompatible glass-ceramics, alumina and zirconia. Calcium phosphate based materials in this list are not active in bone growth and are preferably absent. The amount of such other phases is preferably less than 50% by weight, more preferably less than 3% by weight. However it is preferred in the invention that the granules consist entirely or substantially entirely of the product (e.g. >99% by weight).

In addition, the granular or powder product may be combined with a carrier, such as a hydrogel, a non-aqueous polymer or polymer mixture, or natural organic polymers such as collagens, elastins, carbohydrates or other suitable excipients.

The granular or powder product may be combined with active biomolecules such as growth factor proteins (such as bone morphogenetic proteins), antibiotics (such as gentamicin) or other pharmaceutical drugs, cytokines or antibodies.

The granular or powder product may be combined with cells. This may be done in the operating theatre, immediately prior to implantation, or previously where the cells may be cultured for a period of time on the granules prior to implantation. Such cells include, but are not restricted to, autogenous mesenchymal stem cells, allogenic mesenchyrnal stem cells, osteoblast progenitor cells, osteoblast cells, endothelial cells, and combinations of these.

In the third aspect, a specific embodiment, the calcium-deficient silicate-substituted calcium phosphate apatite phase composition (hereafter “product”) of the invention is obtained or obtainable by the method as described. As explained above, the calcium-deficient silicate-substituted calcium phosphate apatite product has a higher thermal stability due to calcium deficiency which may be achieved by the method which involves contacting the silicate-substituted calcium phosphate apatite phase starting material with the acidic solution.

The calcium-deficient silicate-substituted phosphate composition of the third aspect of the invention may be characterised by a unique chemical composition imparted by the novel method of manufacture. In particular, in some embodiments, the product may comprise an apatite phase having a Ca/P molar ratio of from greater than 2.15 to 2.30 and a Ca/(P+Si) molar ratio of from 1.45 to 1.55. Thus an aspect of the invention is a calcium-deficient silicate-substituted calcium phosphate composition comprising an apatite phase having a Ca/P molar ratio of from greater than 2.15 to 2.30 and a Ca/(P+Si) molar ratio of from 1.45 to 1.55.

The calcium-deficient silicate-substituted calcium phosphate apatite composition of the third aspect is osteoinductive. The product is also osteoconductive.

In some embodiments, the apatite phase of the product has a Ca/P molar ratio of up to 2.3, for example less than 2.30, for example, from 2.20 to 2.30, from 2.22 to 2.30, for example, from 2.22 to 2.28.

In some embodiments, the apatite phase of the product has a Ca/(P+Si) molar ratio of from 1.45 to 1.55, for example from 1.45 to 1.54, or from 1.45 to 1.52.

In some embodiments, the product has a Ca/P molar ratio of up to 2.30, for example less than 2.30, for example from 2.20 to 2.30, for example from 2.22 to 2.28, and a Ca/(P+Si) molar ratio of from 1.45 to 1.55, for example from 1.45 to 1.54, or from 1.45 to 1.52.

To the extent that the product composition contains one or more impurity phases, for example including calcium, the molar ratios of the overall composition may be higher than those noted for the apatite phase of the product composition. Therefore, in some embodiments, the calcium-deficient silicate-substituted calcium phosphate apatite composition comprises a Ca/P molar ratio of from greater than 2.15 up to 2.35. In some embodiments, the calcium-deficient silicate-substituted calcium phosphate apatite composition comprises a Ca/(P+Si) molar ratio of from 1.45 to 1.60.

The material containing the calcium-deficient silicate-substituted calcium phosphate apatite composition of the invention may be substantially phase pure. This means that there are substantially no impurity phases, but consistent with the ASTM 1185 (Standard Specification for Composition of Hydroxylapatite for Surgical Implants), a minimum amount of a hydroxyapatite phase of 95 wt % should be observed, with not more than 5 wt % of an impurity phase observed. In a specific embodiment, only one polycrystalline phase may be seen by X-ray diffraction, with no secondary phases visible in the diffraction pattern. The presence of a single silicate-substituted hydroxyapatite phase, and the quantification of the amount of an impurity phase, can be determined using conventional X-ray diffraction analysis and comparing the obtained diffraction pattern with standard patterns for hydroxyapatite (ASTM F2024—Standard Practice for X-ray Diffraction Determination of Phase Content of Plasma-Sprayed Hydroxyapatite Coatings). The exact diffraction peak positions of the silicate-substituted hydroxyapatite phase show a small shift compared to the diffraction peak positions of a hydroxyapatite standard, as the substitution of silicate for phosphate results in a change in the unit cell parameter. This has previously been reported for small amounts of silicate substitution (e.g. I. R. Gibson et al, J. Biomed. Mater. Res. 44 (1999) 422-428). The amount of silicon or silicate incorporated into a silicate-substituted hydroxyapatite, and the Ca/P molar ratio of a silicate-substituted hydroxyapatite may also be evaluated using chemical analysis techniques, for example, X-Ray Fluorescence (XRF), as mentioned previously.

In some embodiments, the product of the third aspect has a thermal stability such that, after exposure to a temperature of 1250° C., the phase composition of the product is unchanged when evaluated using XRD.

Other preferences for the product set out above with reference to the first aspect apply equally to the third aspect of the invention.

A fourth aspect of the invention is a calcium-deficient silicate-substituted calcium phosphate apatite composition according to the first or third aspect, for use in a method of treatment of the human or animal body by surgery or therapy.

In some embodiments, the method is a method of treatment of the human body by therapy.

In some embodiments, the method is a method of body tissue repair. In some embodiments, the method is a method of repairing or replacing body tissue, for example bone.

In some embodiments, the method comprises the treatment of one or more of bone defects, bone trauma or other skeletal indications. In some embodiments, the method comprises the treatment of a bone fracture.

A fifth aspect of the invention is a method of treatment of a patient using the calcium-deficient silicate-substituted calcium phosphate apatite composition according to the first or third aspect.

In some embodiments, the method is a method of body tissue repair. In some embodiments, the method is a method of repairing or replacing body tissue, for example bone.

In some embodiments, the method comprises the treatment of one or more of bone defects, bone trauma or other skeletal indications. In some embodiments, the method comprises the treatment of a bone fracture.

A sixth aspect of the invention is a medical device comprising a coating which includes a calcium-deficient silicate-substituted calcium phosphate apatite composition of the first or third aspect.

A seventh aspect of the invention is a macroporous ceramic bone graft substitute comprising a calcium-deficient silicate-substituted calcium phosphate apatite composition of the first or third aspect.

An eighth aspect of the invention is the use of an acidic solution to improve the thermal stability of a silicate-substituted calcium phosphate apatite phase.

A ninth aspect of the invention is a method of improving the thermal stability of a silicate-substituted calcium phosphate apatite starting material, comprising contacting the silicate-substituted calcium phosphate apatite starting material with an acidic solution, wherein the silicate-substituted calcium phosphate apatite starting material has a Ca/P molar ratio of from 2.3 to 2.6, and a Ca/(P+Si) molar ratio of from 1.56 to 1.66.

A tenth aspect of the invention is a method of manufacturing a medical device for use as an implant, comprising applying a calcium-deficient silicate-substituted calcium phosphate apatite composition of the first or third aspect to a bioinert substrate.

In some embodiments, the method of the tenth aspect comprises thermally spraying the calcium-deficient silicate-substituted calcium phosphate apatite composition of the first or third aspect onto a surface of the bioinert substrate.

Due to the thermal stability of the calcium-deficient silicate-substituted calcium phosphate apatite composition, the apatite phase purity of the material is preserved during the thermal spraying process thereby producing a medical device with improved properties.

The thermal spraying process may involve passing the calcium-deficient silicate-substituted calcium phosphate apatite composition into a flame before spraying onto the bioinert substrate. In some embodiments, the method comprises heating the calcium-deficient silicate-substituted calcium phosphate apatite composition to a temperature of at least 1000° C. before spraying onto the bioinert substrate. In some embodiments, the flame is a plasma flame.

In some embodiments, the bioinert substrate comprises a medical implant.

In some embodiments, the surface of the bioinert substrate comprises metal or polymer. The calcium-deficient silicate-substituted calcium phosphate apatite composition may be applied onto the metal or polymer surface.

### DESCRIPTION OF THE DRAWINGS

FIG. 1 shows X-ray diffraction patterns of a starting material calcined at 900° C., before incubation, and then after incubation in 5% NH₄Cl immersion solution for 1, 24, 72 or 120 hours and then sintered at 1250° C., formation of a biphasic composition for incubation times of 1 and 24 hours and a single phase hydroxyapatite-like composition for incubation times of 72 and 120 hours.

FIG. 2 shows X-ray diffraction patterns of a starting material of composition Ca₁₀(PO₄)₆₋ₓ(SiO₄)ₓ(OH)₂₋ₓ, where x=0 (hydroxyapatite with no silicate substitution), calcined at 900° C., before incubation, and then after incubation in 5% NH₄Cl immersion solution for 120 hours and sintered at 1250° C., showing no change in phase composition.

FIG. 3 shows X-ray diffraction patterns of a starting material of composition Ca₁₀(PO₄)₆₋ₓ(SiO₄)ₓ(OH)₂₋ₓ, where x=0.3 (silicate substituted hydroxyapatite), calcined at 900° C., before incubation, and then after incubation in 5% NH₄Cl immersion solution for 120 hours and sintered at 1250° C., showing no change in phase composition.

FIG. 4 shows X-ray diffraction patterns of a starting material of composition Ca₁₀(PO₄)₆₋ₓ(SiO₄)ₓ(OH)₂₋ₓ, where x=1.4 (silicate substituted calcium phosphate), calcined at 900° C., before incubation, and then after incubation in 5% NH₄Cl immersion solution for 120 hours and sintered at 1250° C. Before incubation the calcined composition has a diffraction pattern similar to hydroxyapatite, but after the immersion process and sintering at 1250° C. the diffraction patterns correspond to the phase silicocamotite, rather than a hydroxyapatite phase.

FIG. 5 shows X-ray diffraction patterns of a starting material of composition Ca₁₀(PO₄)₆₋ₓ(SiO₄)ₓ(OH)₂₋ₓ, where x=2.0 (silicate substituted calcium phosphate), calcined at 900° C., before incubation, and then after incubation in 5% NH₄Cl immersion solution for 120 hours and sintered at 1250° C. Before incubation the calcined composition has a diffraction pattern similar to hydroxyapatite, and after the immersion process and sintering at 1250° C. the diffraction patterns still corresponds to a hydroxyapatite phase, with much narrower peaks but also a shift in the peak positions suggesting a change in unit cell dimensions.

FIG. 6 shows an SEM image of the microstructure of granules produced by incubating in 5% NH₄Cl immersion solution for 120 hours and sintered at 1250° C.

FIG. 7 shows paraffin histology with tetrachrome stain of granules produced by incubating in 5% NH₄Cl immersion solution for 120 hours and sintered at 1250° C. after implantation in a muscle defect in sheep after 12 weeks, with positive staining of bone forming around and between the granules in dark blue (marked “B”).

### EXAMPLES

Aspects and embodiments of the present invention will now be discussed in the following examples. Further aspects and embodiments will be apparent to those skilled in the art. All documents mentioned in this text are incorporated herein by reference.

### Example 1—Conversion of Calcined Silicate-Substituted Hydroxyapatite Granules in 5% NH₄Cl to Form Non-Sintered New Composition Material

Granules of silicated calcium phosphate, nominally with x=2.0 in the idealised composition Ca₁₀(PO₄)₆₋ₓ(SiO₄)ₓ(OH)₂₋ₓ, were produced by methods described in U.S. Pat. Nos. 8,545,895 and 9,492,585 (the contents of which are incorporated herein by reference in their entirety) as the starting material. Briefly, for the purpose of this example, this involved the dropwise addition of phosphoric acid solution to a calcium hydroxide suspension containing tetraethyl orthosilicate (TEOS), with a Ca/P molar ratio of 2.45 and a Ca/(P+Si) molar ratio of 1.64, maintaining a pH of between 10 and 11. After ageing overnight, the suspension was filtered to remove water and the collected precipitate was dried overnight in an oven at approximately 80° C. The dried filter cake was then broken into small granules and for the purpose of this example a size fraction of granules with dimensions between 1 and 2 mm was collected by sieving, and calcined in a furnace at 900° C. A sample of this calcined material was taken and denoted Comparative Composition 1.

Four identical solutions of aqueous ammonium chloride (NH₄Cl) with concentration of 5% were prepared by adding a defined mass (50 g) of NH₄Cl powder to 1000 mL of water in a volumetric flask and mixing until dissolved; these were referred to as Immersion Solutions A, B, C and D. The pH of the immersion solutions were measured with a calibrated pH meter and recorded.

Granules of Comparative Composition 1 were mixed with each 5% ammonium chloride (NH₄Cl) solution at a ratio of 1:60 (15 g of granules in 900 mL of NH₄Cl solution) and incubated at 37° C. for defined time periods as follows:

| Solution | A | B | C | D |
|---|---|---|---|---|
| Incubation time/hours | 1 | 24 | 72 | 120 |

After incubation, granules from each solution were filtered under vacuum using a Buchner funnel and grade 3 filter paper. The pH of incubation filtrates was recorded and used to calculate the variation of pH relative to the starting pH using Equation 1 below.

Relative pH change=[([pH_{f}]−[pH₀])/pH₀]×100% Equation 1

where pH₀ and pH_{f} are the pH values measured from the NH₄Cl solution before granules were added, and post incubation, respectively.

The granules were then rinsed with distilled water and dried to a constant mass in a drying oven at a temperature of between 60° C. and 80° C. After the drying step, the mass loss of granules was recorded and used to calculate the relative % mass loss using Equation 2.

Relative mass loss=(M₀−M_{f})/M₀×100% Equation 2

where M₀ and M_{f} are the masses of granules before and after incubation, respectively.

The compositions obtained from this process were denoted Composition 1 (1 h incubation), Composition 2 (24 h incubation), Composition 3 (72 h incubation) and Composition 4 (120 h incubation).

Samples of the non-sintered new composition granules of Compositions 1-4 were then characterised with X-ray diffraction (XRD) and X-ray fluorescence spectroscopy (XRF), to assess phase purity and composition, and elemental composition, respectively.

Mass losses from the granules after incubation in the immersion solution ranged from 5 to 8% and showed a trend of increasing mass loss with increased immersion time. The pH of the immersion solution increased after each of the immersion times, reaching a final pH of between 7 and 8, Table 1.

TABLE 1
pH values of solutions before and after granules
were incubated in 5% NH₄Cl solutions.

| | pH of solution | pH of solution | pH |
|---|---|---|---|
| Incubation time | before | after | variation |
| (hours) | incubation, pH₀ | incubation, pH_{f} | (%) |
| 1 h (solution A) | 4.66 | 7.40 | 59 |
| 24 h (solution B) | 4.82 | 7.44 | 54 |
| 72 h (solution C) | 4.63 | 7.95 | 72 |
| 120 h (solution D) | 4.73 | 7.66 | 62 |

The X-ray diffraction patterns of the non-sintered new composition granules of Compositions 1-4 showed comparable diffraction patterns for the granules before immersion and after each of the immersion times; diffraction patterns showed broad diffraction peaks, indicative of a nano-crystalline material, that matched the reference pattern of hydroxyapatite (ICDD 09-432).

Elemental composition (Ca/P, Ca/(P+Si) ratios and wt % Si) obtained from XRF analysis performed on samples incubated in 5% NH₄Cl solutions at various time points are summarised in Table 2 below. The composition of the starting material pre-incubation (Comparative Composition 1) is provided for comparison.

TABLE 2
Ca/P, Ca/(P + Si) molar ratios and wt % Si of samples
incubated in 5% NH₄Cl solutions for various time points.

| Composition | Ca/P | Ca/(P + Si) | wt % Si |
|---|---|---|---|
| Comparative | 2.43 | 1.63 | 5.71 |
| Composition 1 | | | |
| Composition 1 | 2.34 | 1.58 | 5.76 |
| Composition 2 | 2.32 | 1.56 | 5.73 |
| Composition 3 | 2.28 | 1.54 | 5.76 |
| Composition 4 | 2.25 | 1.52 | 5.76 |

Immersion of the starting material granules in the 5% NH₄Cl immersion solution resulted in a significant change in the chemical composition of the starting material. Although the silicon (wt % Si) content of the granules remains relatively unchanged, the Ca/P and the Ca/(P+Si) molar ratios decreased significantly with increased immersion time, achieving a Ca/(P+Si) molar ratio of close to 1.5 after 120 hours (Composition 4). The immersion process has an effect of decreasing the relative calcium content of the granules, with a Ca/(P+Si) molar ratio of 1.52-1.56 after 24 hours (Composition 2) that is far from the Ca/(P+Si) molar ratio that is typical for a silicate-substituted hydroxyapatite of 1.63-1.68.

The non-sintered new composition granules produced by this immersion process can be described as a nano-crystalline silicated calcium-deficient hydroxyapatite. This Example shows that the final composition of the material can be controlled by the incubation time in the immersion solution.

### Example 2—Conversion of Calcined Silicate-Substituted Hydroxyapatite Granules in 5% NH₄Cl to Form Sintered New Composition Material

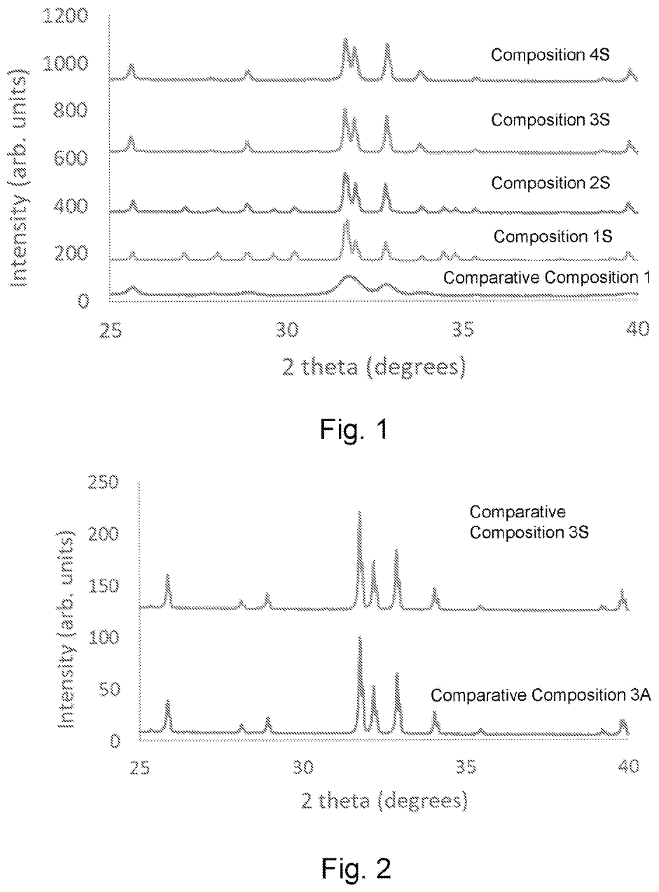

In this Example Compositions 1-4 produced in Example 1 were subjected to a sintering treatment by heating samples in a furnace at temperatures between 900 and 1250° C. Sintering these new compositions will not affect the chemical compositions described in Table 1, but will affect the phase composition. Sintered Compositions 1S, 2S, 3S and 4S were prepared by sintering Compositions 1, 2, 3 and 4 respectively at 1250° C. X-ray diffraction patterns of Compositions 1S, 2S, 3S and 4S are shown in FIG. 1; the calcined sample before incubation is also included for comparison. Incubation times of 1 and 24 hours (Compositions 1S and 2S) resulted in the formation of a biphasic composition, containing silicocamotite and a hydroxyapatite-like phase, but for incubation times of 72 and 120 hours (Compositions 3S and 4S) a diffraction pattern of a single phase hydroxyapatite-like phase was observed; peak positions were shifted compared to the reference pattern of hydroxyapatite (ICDD 09-432), indicative of a change in the unit cell parameters.

The sintered new composition granules of Compositions 1S, 2S, 3S and 4S produced by this immersion process can be described as a crystalline silicated calcium-deficient hydroxyapatite. The final composition, specifically the conditions to produce a single phase hydroxyapatite-like phase, can be controlled by the incubation time in the immersion solution.

### Example 3—Conversion of Non-Calcined Silicate-Substituted Hydroxyapatite Granules in 5% NH₄Cl to Form Non-Sintered or Sintered New Composition Material

Granules were produced in a similar way to those described in Example 1, but the granules were not calcined prior to incubating in the immersion solution (i.e. the step of calcining the granules at 900° C. was omitted). Non-calcined granules were incubated in 5% NH₄Cl immersion solution for 120 hours, then samples were collected by filtration, washed with water then dried in an oven at a temperature of between 60° C. and 80° C., to provide a composition which was denoted Composition 5. Elemental composition (Ca/P, Ca/(P+Si) ratios and wt % Si) obtained from XRF analysis and phase composition analysis by XRD of Composition 5 showed a similar phase composition and chemical composition to that observed in Compositions 1-4. Composition 5 was then sintered at 1250° C. to provide sintered granules denoted Composition 5S; XRD analysis showed that the same single phase hydroxyapatite-like composition was formed in Composition 5S as was observed in Compositions 3S and 4S of Example 2.

The results showed that the initial calcination of the starting material before incubation did not significantly affect the chemical and phase composition of the new composition after incubation in the 5% NH₄Cl immersion solution for 120 hours, both non-sintered or after sintering at 1250° C.

### Example 4—Effect of the Form of the Starting Material on the Conversion of Calcined Silicate-Substituted Hydroxyapatite Granules in 5% NH₄Cl to Form Non-Sintered or Sintered New Composition Material

Filter cake was produced as described in Example 1, but it was not granulated, rather it was calcined at 900° C. as a monolith filter cake. Calcined filter cake was then incubated in 5% NH₄Cl immersion solution for 120 hours and compared to the incubated calcined granules from Example 1 (Composition 4). Samples were collected by filtration, washed with water then dried in an oven at a temperature of between 60° C. and 80° C. Elemental composition (Ca/P, Ca/(P+Si) ratios and wt % Si) obtained from XRF analysis performed on samples incubated in 5% NH₄Cl solutions at various time points are summarised in Table 3 below.

TABLE 3
Ca/P, Ca/(P + Si) molar ratios and wt % Si of samples
with starting material in the form of calcined granules or calcined
filtercake incubated in 5% NH₄Cl solution for 120 hours.

| Form of the | | | |
|---|---|---|---|
| starting material | Ca/P | Ca/(P + Si) | wt % Si |
| Granules | 2.25 | 1.52 | 5.76 |
| (Composition 4) | | | |
| Filtercake | 2.25 | 1.52 | 5.90 |

The form that the starting material was in did not significantly affect the new composition after incubation in the 5% NH₄Cl immersion solution for 120 hours.

### Example 5—Effect of NH₄Cl Immersion Solution Concentration on the Conversion of Non-Calcined Silicate Substituted HA Granules to Form Non-Sintered or Sintered New Composition Material

The effect of the concentration of the NH₄Cl immersion solution on the conversion of calcined silicate-substituted HA granules to form new composition material was studied using an incubation time of 24 hours. Calcined starting material granules were incubated in NH₄Cl immersion solutions as described in Example 1, with a range of NH₄Cl solution concentrations from 0 (water), 0.01%, 1%, 5% and 10%, for a period of 24 hours. Granules were collected by filtration, washed with water, then dried in an oven at a temperature of between 60° C. and 80° C.

The compositions obtained by this process were denoted Comparative Composition 2 (incubation in water alone), Composition 6 (incubation in 0.01% NH₄Cl solution), Composition 7 (incubation in 1% NH₄Cl solution), Composition 8 (incubation in 5% NH₄Cl solution) and Composition 9 (incubation in 10% NH₄Cl solution).

The chemical composition of the material was then determined by XRF analysis; the results presented as Ca/P, Ca/(P+Si) molar ratios and wt % Si are presented in Table 4. As for the effect of incubation time in Example 1, the silicon content (wt % Si) of the new composition materials was not significantly affected by the concentration of the NH₄Cl immersion solution. The Ca/P and the Ca/(P+Si) molar ratios decreased significantly with increased concentration of the NH₄Cl immersion solution, although a concentration of 1% was required to result in a significant decrease in the Ca/P molar ratio (Composition 7) and concentration of 5% was required to result in a significant decrease in the Ca/(P+Si) molar ratio (Composition 8). The concentration of the NH₄Cl immersion solution has an effect of decreasing the relative calcium content of the granules, with a Ca/(P+Si) molar ratio of 1.50-1.56 after 24 hours for 5 and 10% NH₄Cl immersion solution (Compositions 8 and 9) that is far from the Ca/(P+Si) molar ratio that is typical for a silicate-substituted hydroxyapatite of 1.63-1.68.

TABLE 4
Ca/P, Ca/(P + Si) molar ratios and wt % Si of samples
incubated for 24 hours in 0.01, 1, 5, 10% NH₄Cl solutions.

| Composition | Ca/P | Ca/(P + Si) | wt % Si |
|---|---|---|---|
| Comparative | 2.43 | 1.63 | 5.71 |
| Composition 1 | | | |
| Comparative | 2.41 | 1.61 | 5.71 |
| Composition 2 | | | |
| (immersion in water) | | | |
| Composition 6 | 2.40 | 1.60 | 5.72 |
| Composition 7 | 2.35 | 1.59 | 5.67 |
| Composition 8 | 2.32 | 1.56 | 5.73 |
| Composition 9 | 2.22 | 1.50 | 5.76 |

The non-sintered new composition granules of Compositions 6-9 produced by this immersion process can be described as a nano-crystalline silicated calcium-deficient hydroxyapatite. This Example shows that the final composition can be controlled by the concentration of acid in the immersion solution and/or the incubation time. For example, Composition 9, treated with 10% ammonium chloride for 24 hours and resulting in a chemical composition with Ca/P=2.22 and Ca/(P+Si)=1.50, was comparable with Composition 4 from Example 1, treated with 5% ammonium chloride for 120 hours and resulting in a chemical composition with Ca/P=2.25 and Ca/(P+Si)=1.52. Both treatments provided a calcium-deficient silicate-substituted calcium phosphate composition.

### Example 6—Effect of the Chemical Composition of the Starting Material on the Conversion to a New Composition Material by Incubating in an Immersion Solution

The effect of the composition of the starting material used on the composition of the material after incubation in the immersion solution was studied by incubating starting materials with various values of x (0, 0.3, 1.4 and 2) in the idealised composition Ca₁₀(PO₄)₆₋ₓ(SiO₄)ₓ(OH)₂₋ₓ in 5% NH₄Cl immersion solution for 120 hours. Each of the starting material compositions were synthesised using a similar process to that described in Example 1, except the relative amounts of reagents were varied in accordance with the final desired composition. Samples of the compositions after calcination at 900° C. but before immersion in any solution were also taken and analysed; these were denoted Comparative Composition 3A (x=0), Comparative Composition 4A (x=0.3), Comparative Composition 5A (x=1.4). Comparative Composition 1 from Example 1 was used for the x=2.0 pre-immersion sample.

Starting material granules that had been calcined at 900° C. were incubated in 5% NH₄Cl immersion solutions as described in Example 1, for a period of 120 hours. Granules were collected by filtration, washed with water, then dried in an oven at a temperature of between 60° C. and 80° C.

Composition 4 prepared in Example 1 was used as the x=2.0 sample. The new compositions prepared in the present Example were denoted Comparative Composition 3B (x=0), Comparative Composition 4B (x=0.3) and Comparative Composition 5B (x=1.4).

The chemical composition of the materials were then determined by XRF analysis; the results presented as Ca/P, Ca/(P+Si) molar ratios and wt % Si are presented in Table 5.

Of note, the compositions with no silicon in the starting material (Comparative Composition 3A) or a low level of silicon (x=0.3, or approximately 0.8 wt % Si; Comparative Composition 4A) were unaffected by the immersion process. This is important as Comparative Composition 3A corresponds to hydroxyapatite which has been studied for over 40 years as a bone replacement material, and Comparative Composition 4A corresponds to a silicate-substituted hydroxyapatite composition that has been studied as a bone replacement material for over 20 years. The immersion process described here clearly does not have a significant effect on the chemical composition of these two starting materials.

For a starting material with a composition of x=1.4 (Comparative Composition 5A), the immersion process in 5% NH₄Cl for 120 hours had a similar effect to the starting material with a composition of x=2.0 (Comparative Composition 1), with a decrease in the Ca/P and the Ca/(P+Si) molar ratios, resulting in a new composition (Comparative Composition 5B), although the Ca/(P+Si) molar ratio does not approach a value close to 1.5 as was observed with x=2.0 for incubation times of 72-120 hours for 5% NH₄Cl (Compositions 3 and 4, Example 1), or 24 hours for 10% NH₄Cl (Composition 9, Example 5).

TABLE 5
Ca/P, Ca/(P + Si) molar ratios and wt % Si of samples
incubated for 120 hours in 5% NH₄Cl solution, produced with
starting materials of different composition; the value of x was varied
from 0 to 2 in the idealised composition Ca₁₀(PO₄)₆₋ₓ(SiO₄)ₓ(OH)₂₋ₓ

| | Value of x in | | Ca/ | wt % |
|---|---|---|---|---|
| Composition | Ca₁₀(PO₄)₆₋ₓ(SiO₄)ₓ(OH)₂₋ₓ] | Ca/P | (P + Si) | Si |
| Comparative Composition 3A | 0 | 1.69 | 1.69 | 0 |
| (starting material) | | | | |
| Comparative Composition 3B | 0 | 1.70 | 1.70 | 0 |
| (incubated) | | | | |
| Comparative Composition 4A | 0.3 | 1.74 | 1.66 | 0.74 |
| (starting material) | | | | |
| Comparative Composition 4B | 0.3 | 1.74 | 1.67 | 0.71 |
| (incubated) | | | | |
| Comparative Composition 5A | 1.4 | 2.15 | 1.65 | 3.98 |
| (starting material) | | | | |
| Comparative Composition 5B | 1.4 | 2.03 | 1.60 | 3.67 |
| (incubated) | | | | |
| Comparative Composition 1 | 2.0 | 2.43 | 1.63 | 5.71 |
| Composition 4 | 2.0 | 2.25 | 1.52 | 5.76 |
| (incubated) | | | | |

The effect of the starting material composition on the phase composition of the material produced after the immersion process was negligible, with the diffraction patterns of Comparative Compositions 3B, 4B, 5B and 4 and Comparative Composition 1 showing no significant difference in the patterns.

The new composition granules produced by this immersion process can be described as a nano-crystalline silicated calcium-deficient hydroxyapatite. The final composition of the new composition granules can be controlled by the chemical composition of the starting material subjected to the immersion process for certain compositions, whereas some starting material composition, such as x=0 or x=0.3, are largely unaffected by the immersion process.

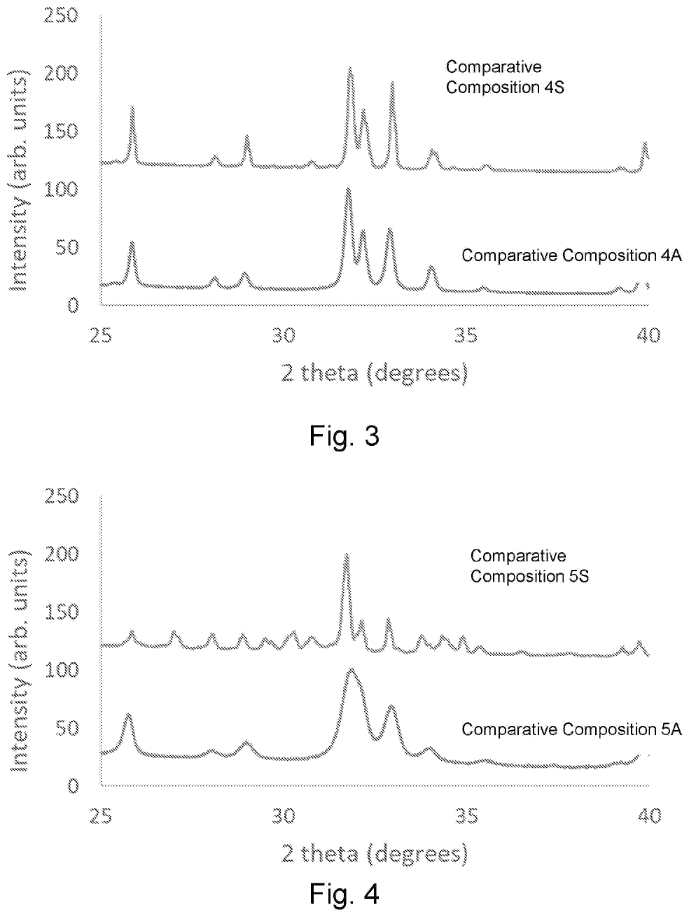

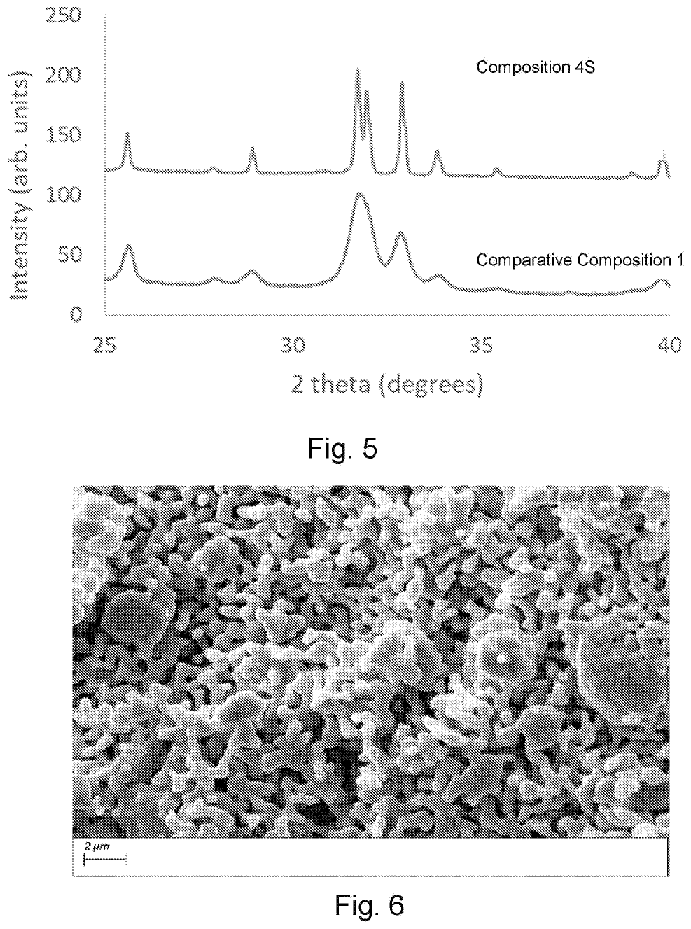

Comparative Composition 3B, Comparative Composition 4B and Comparative Composition 5B were then sintered at 1250° C. to prepare Comparative Composition 3S, Comparative Composition 4S and Comparative Composition 5S respectively. These sintered compositions and sintered Composition 4S from Example 2 were studied with XRD. Sintering the various compositions at 1250° C. after the immersion treatment had a notable effect on some compositions. For the two compositions with no silicon in the starting material (Comparative Composition 3B) or a low level of silicon (Comparative Composition 4B), the phase composition and diffraction peak shape remained unchanged after sintering at 1250° C., with Comparative Compositions 3S and 4S showing sharp diffraction peaks that matched the reference pattern of hydroxyapatite (ICDD 09-432), FIGS. 2 and 3. For Comparative Composition 5A (x=1.4), the diffraction pattern showed a pattern similar to hydroxyapatite, FIG. 4, similar to Comparative Composition 1 (x=2.0), FIG. 5, but after the immersion process and sintering at 1250° C., the diffraction pattern (Comparative Composition 5S) corresponded to the phase silicocamotite, rather than a hydroxyapatite phase. For the calcined starting material with x=2.0, after the immersion process and sintering at 1250° C., the diffraction pattern still corresponds to a hydroxyapatite phase, with much narrower peaks but also a shift in the peak positions suggesting a change in unit cell dimensions.

This data confirms that the immersion process has little effect on starting materials with no or only small amounts of silicon substitution (x=0 and x=0.3), but for higher levels of silicon substitution (x=2.0), the immersion process changes the chemical composition significantly and results in the high thermal stability of a hydroxyapatite-like phase after sintering at 1250° C. Intermediate compositions, such as x=1.4, do undergo a change in chemical composition after the immersion process but the phase composition after sintering at 1250° C. does not produce a hydroxyapatite-like phase, but rather results in the formation of the phase silicocamotite.

### Example 7—Effect of the Phase Composition of the Starting Material on the Conversion to a New Composition Material by Incubating in an Immersion Solution

Synthesis of calcium phosphates by methods such as aqueous precipitation sometimes results in products that contain small amounts of impurity phases which may affect the properties of the target phase composition. The feasibility of using starting material that contains a small amount of phase impurities, that could be considered as “out of specification” batches by conventional hydroxyapatite standards, to form the new composition product, was assessed. Two compositions similar to that described in Example 1 were prepared, but with a deficiency (Composition 11) and an excess (Composition 12) of calcium in the reaction mixture, respectively. The precipitated suspension was processed to calcined granules in a similar manner to the granules in Example 1, resulting in impurity phases of tricalcium phosphate or calcium oxide for the deficiency or excess of calcium in the reaction mixture, respectively. Samples of the materials were taken after initial calcination but before immersion in NH₄Cl solution, as Comparative Composition 11 (calcium deficiency) and Comparative Composition 12 (calcium excess).

The granules were then incubated in 5% NH₄Cl immersion solution for 120 hours in a similar manner. Samples were collected by filtration, washed with water then dried in an oven at a temperature of between 60° C. and 80° C. to provide Compositions 11 and 12. Elemental composition (Ca/P, Ca/(P+Si) ratios and wt % Si) obtained from XRF analysis are summarised in Table 6 below.

TABLE 6
Ca/P, Ca/(P + Si) molar ratios and wt % Si of samples from
starting materials with different phase compositions before incubation
and after incubation in 5% NH₄Cl solutions for 120 hours.

| | | | Ca/(P + | |
|---|---|---|---|---|
| Composition | | Ca/P | Si) | wt % Si |
| Comparative Composition 1 | | 2.43 | 1.63 | 5.71 |
| (Example 1) | | | | |
| Composition 4 (Example 1) | | 2.25 | 1.52 | 5.76 |
| Ca-deficient compositions | Comparative | 2.38 | 1.60 | 5.76 |
| | Composition 11 | | | |
| | Composition 11 | 2.24 | 1.52 | 5.69 |
| Ca-rich compositions | Comparative | 2.49 | 1.65 | 5.77 |
| | Composition 12 | | | |
| | Composition 12 | 2.28 | 1.53 | 5.84 |

The Ca/P and Ca/(P+Si) molar ratios of the starting material before incubation from Example 1 (Comparative Composition 1) falls between the values for the Ca-deficient composition (Comparative Composition 11) and the Ca-rich composition (Comparative Composition 12), whereas the silicon contents (wt % Si) were comparable. Incubation in the 5% NH₄Cl immersion solution for 120 hours resulted in a decrease in the Ca/P and Ca/(P+Si) molar ratios of all the products to very comparable values. This shows that the immersion process can utilise starting materials that contain an excess or a deficiency of calcium but that after the incubation in the immersion solution, a similar chemical composition can be obtained. This was confirmed by XRD analysis of the compositions produced by the immersion process and sintered to 1250° C., where diffraction patterns of a single phase hydroxyapatite-like phase similar to that in FIG. 5 in Example 6 were observed.

### Example 8—Effect of Properties of Material after Conversion and Heat Treatment—Surface Area and Porosity and Osteoinductivity

The microstructure of the granules of Composition 4S produced in Example 2 was analysed using SEM. The surface area of the granules was measured by nitrogen gas adsorption using the BET Method (ASTM C1274-12(2020), Standard Test Method for Advanced Ceramic Specific Surface Area by Physical Adsorption). The microstructure shows fused/sintered granules approximately 1-2 μm in size, with significant porosity between areas of fused granules; some regions of more densely sintered granules were evident, FIG. 6. The specific surface area of the granules was measured as about 3 m²/g.

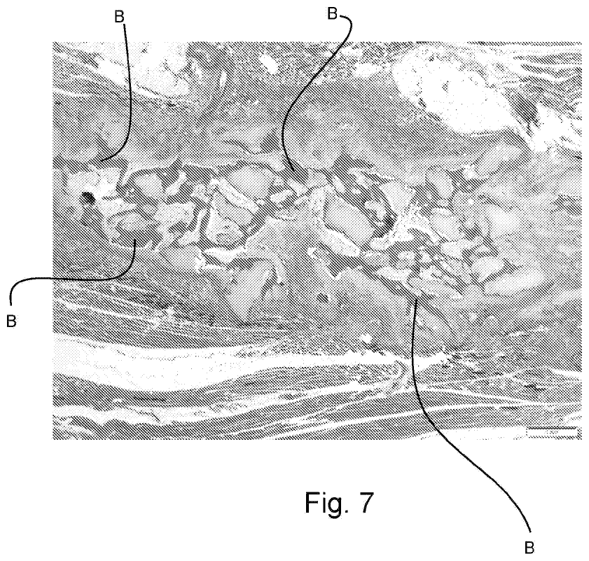

To assess the ability of the new composition to induce the formation of bone in vivo (osteoinduction), 1 cc of the granules of Composition 4S were implanted into muscle defects in sheep. After 12 weeks the explants were fixed, decalcified, embedded in paraffin, and cut into histological sections and stained using a tetrachrome stain that stains new bone/osteoid as a deep blue colour. A representative histology section is shown in FIG. 7 and shows the formation of new bone around and between the granules, confirming that granules of the new composition are osteoinductive. Some of the areas of new bone formation, which appear deep blue in the original SEM image, are marked with the letter “B” in FIG. 7. The scale bar in the bottom right corner of the SEM image of FIG. 7 is 1 mm.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A calcium-deficient silicate-substituted calcium phosphate apatite composition comprising an apatite phase having a Ca/P molar ratio of from greater than 2.15 to 2.30, and a Ca/(P+Si) molar ratio of from 1.45 to 1.55.

2. The composition according to claim 1, wherein the apatite phase has a Ca/P molar ratio of from greater than 2.15 to 2.28, from 2.20 to 2.30, or from 2.20 to 2.28.

3. The composition according to claim 1, wherein the apatite phase has a Ca/(P+Si) molar ratio of from 1.45 to 1.54, or from 1.45 to 1.52.

4. The composition according to claim 1, having a silicon content of 4 to 6 wt %.

5. The composition according to claim 1, densified by sintering at a temperature of from 1100 to 1300° C.

6. The composition according to claim 1, comprising up to 5 wt % total of a phase or phases other than the apatite phase, and the composition has Ca/P molar ratio of from greater than 2.15 to 2.35, and a Ca/(P+Si) molar ratio of from 1.45 to 1.60.

7. The composition according to claim 1, wherein the composition consists or consists essentially of the apatite phase.

8. A method of producing a calcium-deficient silicate-substituted calcium phosphate apatite composition, comprising contacting a silicate-substituted calcium phosphate apatite starting material with an acidic solution to produce the calcium-deficient silicate-substituted calcium phosphate apatite composition,
wherein the starting material comprises an apatite phase and has a Ca/P molar ratio of from 2.3 to 2.6, and a Ca/(P+Si) molar ratio of from 1.56 to 1.66, and
wherein the calcium-deficient silicate-substituted calcium phosphate apatite composition comprises an apatite phase having a Ca/P molar ratio which is lower than the Ca/P ratio of the starting material apatite phase before contact with the acidic solution.

9. A method according to claim 8, wherein the starting material comprises a silicon atom content of from 4 to 6 wt %.

10. A method according to claim 8, wherein the starting material comprises up to 15 wt % total of a phase or phases other than the apatite phase.

11. A method according to claim 8, wherein the starting material comprises a material according to formula (I):
Ca_{10-δ}(PO₄)₆₋ₓ(SiO₄)ₓ(OH)_{2-y} (I)
wherein 1.1≤x≤2.0, 1.0≤y 2.0, and δ represents a Ca deficiency.

12. A method according to claim 8, wherein the starting material is a powder with a specific surface area of from 10 to 90 m²/g.

13. A method according to claim 8, wherein the starting material is a powder with a Dᵥ50 less than 100 μm, or comprises granules having an average particle diameter Dᵥ50 greater than 100 μm.

14. A method according to claim 8, wherein the acidic solution is an aqueous acidic solution.

15. A method according to claim 8, wherein the acidic solution comprises an acid component and a liquid vehicle, wherein the acid component is an acid having a pKa of greater than −1.73.

16. A method according to claim 8, wherein the acidic solution comprises or consists of an aqueous ammonium chloride solution.

17. A method according to claim 16, wherein the aqueous ammonium chloride solution has an ammonium chloride concentration of from 0.01% w/v to 15% w/v.

18. A method according to claim 8, comprising mixing the acidic solution and the starting material in a weight ratio of at least 5:1.

19. A method according to claim 8, comprising incubating the mixture of the starting material and the acidic solution for a predetermined period of time.

20. A method according to claim 19, wherein incubating the mixture comprises heating the incubation mixture to a temperature T₁ and allowing the incubation mixture to remain at temperature T₁ for a time t₁, wherein T₁ is at least 30° C. and t₁ is at least 10 mins.

21. A method according to claim 8, comprising separating the calcium-deficient silicate-substituted calcium phosphate apatite composition from the acidic solution.

22. A method according to claim 8, further comprising one or more steps of sintering the calcium-deficient silicate-substituted calcium phosphate apatite composition at a temperature of at least 100° C.

23. A method according to claim 8, wherein the calcium-deficient silicate-substituted calcium phosphate apatite composition comprises a Ca/(P+Si) molar ratio which is lower than the Ca/(P+Si) ratio of the starting material before contact with the acidic solution.

24. A method according to claim 8, wherein the calcium-deficient silicate-substituted calcium phosphate apatite composition comprises a Ca/P molar ratio of from greater than 2.15 to 2.35 and a Ca/(P+Si) molar ratio of from 1.45 to 1.60.

25. A method according to claim 8, wherein the calcium-deficient silicate-substituted calcium phosphate apatite composition comprises a silicon atom content of from 4 to 6 wt %.

26. A method according to claim 8, wherein the calcium-deficient silicate-substituted calcium phosphate apatite composition comprises an apatite phase having a Ca/P molar ratio of from greater than 2.15 to 2.30, and a Ca/(P+Si) molar ratio of from 1.45 to 1.55.

27. A calcium-deficient silicate-substituted calcium phosphate apatite composition obtained or obtainable by a method according to claim 8.

28.-29. (canceled)

30. A medical device comprising a coating which includes a calcium-deficient silicate-substituted calcium phosphate apatite composition according to claim 1.

31. A macroporous ceramic bone graft substitute comprising a calcium-deficient silicate-substituted calcium phosphate apatite composition according to claim 1.

32. (canceled)

33. A method of treating a disease or disorder requiring the replacement of bone tissue, comprising replacing bone tissue with a calcium-deficient silicate-substituted calcium phosphate apatite composition according to claim 1.

34. A method according to claim 8, wherein the acidic solution improves the thermal stability of the calcium-deficient silicate-substituted calcium phosphate apatite phase.
